(19) United States
(12) Patent Application Publication
(10) Pub. No.: US 2008/0139915 A1
(43) Pub. Date: Jun. 12, 2008

(54) Vascular Position Locating and/or Mapping Apparatus and Methods

(75) Inventors: Mark Dolan, Santa Rosa, CA (US); David Simon, Boulder, CO (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/695,160

(22) Filed: Apr. 2, 2007

Related U.S. Application Data
(63) Continuation-in-part of application No. 11/608,081, filed on Dec. 7, 2006.

(51) Int. Cl. A61B 5/05; A61F 2/06

(52) U.S. Cl. 600/407; 600/13; 600/417; 623/1.18

(57) ABSTRACT

A branch vessel in a human patient is located or mapped using in vivo tracked field sensors where in one variation the sensor positions can be located by determining the positions of the sensors relative to a plurality of magnetic field sources of known location. This approach is used, for example, in locating the opening in a renal artery and positioning the proximal end of the AAA stent-graft adjacent to the opening. In another example, the sensors are tracked along the inner wall of an aneurysm and the acquired sensor location data processed to map the contour of the aneurysm to size a prostheses for spanning the aneurysm. The portions of the vessel adjacent the aneurysm also can be mapped. In a further embodiment, an in vivo sensor is positioned in a deployed prosthesis to create a reference for a prosthetic member having a sensor to track to during cannulation of the deployed prosthesis with the prosthetic member.

### CROSS-REFERENCE

This application is a continuation-in-part application of Ser. No. 11/608,081, filed Dec. 7, 2006 and entitled Vascular Position Locating Apparatus and Methods, which application is incorporated herein by reference in its entirety and to which application we claim priority under 35 USC §120.

### FIELD OF THE INVENTION

The invention relates to prosthesis deployment and more particularly to locating a branch passageway in a human body such as a branch artery prior to prosthesis deployment or locating a passageway in a prosthesis prior to in vivo cannulation thereof.

### BACKGROUND OF THE INVENTION

Tubular prostheses such as stents, grafts, and stent-grafts (e.g., stents having an inner and/or outer covering comprising graft material and which may be referred to as covered stents) have been widely used in treating abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE)) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel. The abnormally dilated vessel has a wall that typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft spans across and extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened, and a graft is sutured into position bypassing the aneurysm. The endovascular approach, which has been used to deliver stents, grafts, and stent grafts, generally involves cutting through the skin to access a lumen of the vasculature. Alternatively, lumenar or vascular access may be achieved percutaneously via successive dilation at a less traumatic entry point. Once access is achieved, the stent-graft can be routed through the vasculature to the target site. For example, a stent-graft delivery catheter loaded with a stent-graft can be percutaneously introduced into the vasculature (e.g., into a femoral artery) and the stent-graft delivered endovascularly to a portion where it spans across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter and self expands upon retraction or removal of the sheath at the target site. More specifically, a delivery catheter having coaxial inner and outer tubes arranged for relative axial movement there between can be used and loaded with a compressed self-expanding stent-graft. The stent-graft is positioned within the distal end of the outer tube (sheath) and in front of a stop fixed to distal end of the inner tube. Regarding proximal and distal positions referenced herein, the proximal end of a prosthesis (e.g., stent-graft) is the end closest to the heart (by way of blood flow) whereas the distal end is the end furthest away from the heart during deployment. In contrast, the distal end of a catheter is usually identified as the end that is farthest from the operator, while the proximal end of the catheter is the end nearest the operator. Once the catheter is positioned for deployment of the stent-graft at the target site, the inner tube is held stationary and the outer tube (sheath) withdrawn so that the stent-graft is gradually exposed and expands. An exemplary stent-graft delivery system is described in U.S. Patent Application Publication No. 2004/0093063, which published on May 13, 2004 to Wright et al. and is entitled Controlled Deployment Delivery System, the disclosure of which is hereby incorporated herein in its entirety by reference.

Although the endovascular approach is much less invasive, and usually requires less recovery time and involves less risk of complication as compared to open surgery, there can be concerns with alignment of asymmetric features of various prostheses in relatively complex applications such as one involving branch vessels. Branch vessel techniques have involved the delivery of a main device (e.g., a graft or stent-graft) and then a secondary device (e.g., a branch graft or branch stent-graft) through a fenestration or side opening in the main device and into a branch vessel.

The procedure becomes more complicated when more than one branch vessel is treated. One example is when an aortic abdominal aneurysm is to be treated and its proximal neck is diseased or damaged to the extent that it cannot support a reliable connection with a prosthesis. In this case, grafts or stent-grafts have been provided with fenestrations or openings formed in their side wall below a proximal portion thereof. The fenestrations or openings are to be aligned with the renal arteries and the proximal portion is secured to the aortic wall above the renal arteries.

To ensure alignment of the prostheses fenestrations and branch vessels, some current techniques involve placing guidewires through each fenestration and branch vessel (e.g., artery) prior to releasing the main device or prosthesis. This involves manipulation of multiple wires in the aorta at the same time, while the delivery system and stent-graft are still in the aorta. In addition, an angiographic catheter, which may have been used to provide detection of the branch vessels and preliminary prosthesis positioning, may still be in the aorta. Not only is there risk of entanglement of these components, the openings in an off the shelf prosthesis with preformed fenestrations may not properly align with the branch vessels due to differences in anatomy from one patient to another. Prostheses having preformed custom located fenestrations or openings based on a patient's CAT scans also are not free from risk. A custom designed prosthesis is constructed based on a surgeon's interpretation of the scan and still may not result in the desired anatomical fit. Further, relatively stiff catheters are used to deliver grafts and stent-grafts and these catheters can apply force to tortuous vessel walls to reshape the vessel (e.g., artery) in which they are introduced. When the vessel is reshaped, even a custom designed prosthesis may not properly align with the branch vessels.

U.S. Pat. No. 5,617,878 to Taheri discloses a method comprising interposition of a graft at or around the intersection of major arteries and thereafter, use of intravenous ultrasound or angiogram to visualize and measure the point on the graft where the arterial intersection occurs. A laser or cautery device is then interposed within the graft and used to create an opening in the graft wall at the point of the intersection. A stent is then interposed within the graft and through the created opening of the intersecting artery.

U.S. Patent Application Ser. No. 11/276,512 to Marilla, entitled Multiple Branch Tubular Prosthesis and Methods, filed Mar. 3, 2006, and co-owned by the assignee of the present application discloses positioning in an endovascular prosthesis an imaging catheter (intravenous ultrasound device (IVUS)) having a device to form an opening in the side wall of the prosthesis. The imaging catheter detects an area of the prosthesis that is adjacent to a branch passageway (e.g., a renal artery), which branches from the main passageway in which the prosthesis has been deployed. The imaging catheter opening forming device is manipulated or advanced to form an opening in that area of the prosthesis to provide access to the branch passageway. The imaging catheter also can include a guidewire that can be advanced through the opening.

Generally speaking, one challenge in prosthesis (e.g., stent graft) delivery/placement in the vicinity of one or more branch vessels is identifying and locating the position of branch vessels (e.g., arteries). Typically fluoroscopy is used to identify branch vessels. More specifically, fluoroscopy has been used to observe real-time X-ray images of the openings within cardiovascular structures such as the renal arteries during a stent-graft procedure. This approach requires one to administer a radiopaque substance, which generally is referred to as a contrast medium, agent or dye, into the patient so that it reaches the area to be visualized (e.g., the renal arteries). A catheter can be introduced through the femoral artery in the groin of the patient and endovascularly advanced to the vicinity of the renals. The fluoroscopic images of the transient contrast agent in the blood, which can be still images or real-time motion images, allow two dimensional visualization of the location of the renals.

The use of X-rays, however, requires that the potential risks from a procedure be carefully balanced with the benefits of the procedure to the patient. While physicians always try to use low dose rates during fluoroscopy, the duration of a procedure may be such that it results in a relatively high absorbed dose to the patient. Patients who cannot tolerate contrast enhanced imaging or physicians who must or wish to reduce radiation exposure need an alternative approach for defining the vessel configuration and branch vessel location.

Accordingly, there remains a need to develop and/or improve prosthesis deployment apparatus and methods for endoluminal or endovascular applications.

### SUMMARY OF THE INVENTION

The present invention involves improvements in prosthesis deployment apparatus and methods.

In one embodiment according to the invention, a method of locating a branch vessel in a human patient comprises tracking a sensor moving or being navigated in a vessel along a first path (e.g., along a portion of a vessel wall); and detecting movement of the sensor away from the path (e.g., generally orthogonal to the path). The detected movement can be evaluated or monitored to confirm if branch vessel detection occurred.

In another embodiment according to the invention, a method of positioning a tubular prosthesis in a passageway in a human body comprises advancing a tubular prosthesis through a vessel in a patient; obtaining the position in three dimensions of a portion of an opening to a branch vessel; and positioning the proximal end portion of the prosthesis at a predetermined distance from the branch vessel opening portion. In one example, the vessel can be the aorta of the patient and the branch vessel can be a renal artery.

In another embodiment according to the invention, a method of cannulating a bifurcated tubular prosthesis in vivo comprises positioning a bifurcated tubular prosthesis in the aorta of a patient having an ipsilateral leg and a truncated contralateral leg portion; positioning a first sensor in the truncated contralateral leg portion; obtaining the position in three dimensions of the first sensor; advancing a contralateral leg delivery catheter, which has a distal portion and a proximal portion and a second sensor coupled to the distal portion, toward the first sensor position; and monitoring the second sensor position in three dimensions relative to the first sensor position to guide the distal portion of the contralateral leg delivery catheter into the truncated contralateral leg portion.

In another embodiment according to the invention, a prosthesis delivery system comprises a stent-graft delivery catheter having a proximal end portion and a distal end portion; a first sensor coupled to the catheter distal end portion; a flexible member having a fixed end portion and a feeler end portion, the flexible member fixed end portion being secured to the catheter distal end portion; and a second signal sensor coupled to the flexible member feeler end portion and suspended thereby.

In another embodiment according to the invention, a prosthesis delivery system comprises a tubular prosthesis delivery sheath having a proximal end portion and a distal end portion; a tip member having a proximal end portion and a distal end portion, the tip member proximal end portion being releasably coupled to the sheath distal end portion; a first sensor coupled to the tip member; a flexible member having a fixed end portion and a feeler end portion, the flexible member fixed end portion being secured to the tip member; and a second sensor coupled to the flexible member and suspended thereby.

In another embodiment according to the invention, a stent-graft delivery system comprises a stent-graft delivery catheter having a proximal end portion and a distal end portion; a flexible member having a fixed end portion and a feeler end portion, the flexible member fixed end portion being secured to the catheter distal end portion; a first sensor coupled to one of the catheter distal end portion and the flexible member; a signal generator coupled to the other of the catheter distal end portion and the flexible member; and the one of the sensor and signal generator that is coupled to the flexible member being suspended thereby.

In another embodiment according to the invention, a stent-graft delivery system comprises a stent-graft delivery sheath having a proximal end portion and a distal end portion; a tip member having a proximal end portion and a distal end portion, the tip member being releasably coupled to the sheath distal end portion; a flexible member having a fixed end portion and a feeler end portion, the flexible member fixed end portion being secured to the tip member; a first sensor coupled to one of the tip member and the flexible member; a signal generator coupled to the other of the tip member and the flexible member; and the one of the sensor and signal generator that is coupled to the flexible member being suspended thereby and movable relative to the tip member.

In another embodiment according to the invention, a probe for locating or mapping structure in a patient comprises an elongated member configured for endovascular delivery in a patient, the elongated member having a proximal end portion and a distal end portion; a first sensor coupled to the elongated member distal end portion; a flexible member having a first portion and a second portion, the flexible member first portion being coupled to the elongated member distal end portion; and a second sensor attached to the flexible member and suspended thereby.

In another embodiment according to the invention, a method of mapping the contour of an inner surface of a vessel wall in a patient comprises advancing a plurality of sensors along an inner surface of a vessel wall in a patient; acquiring data indicative of the position of the sensors in three-dimensional space as they are advanced along the surface; and processing the acquired data to generate a three-dimensional image corresponding to the contour of a portion of the inner vessel surface.

In another embodiment according to the invention, a method of mapping the contour of an inner surface of a vessel wall in a patient comprises advancing a sensor along the inner surface of a vessel wall in a patient in both a circumferential and axial direction; acquiring data indicative of the position of the sensor in three dimensional space as it is advanced along the surface; and processing the acquired data to generate a three-dimensional image corresponding to the contour of a portion of the inner vessel surface.

In another embodiment according to the invention, a method of selecting vascular prosthesis comprises advancing a sensor along an inner surface of a vessel wall; acquiring data indicative of the position of the sensor in three-dimensional space as it is advanced along the inner surface; and selecting a prosthesis based on the acquired data.

Other features, advantages, and embodiments according to the invention will be apparent to those skilled in the art from the following description and accompanying drawings.

### BRIEF DESCRIPTION OF THE DRAWINGS

FIG. 1 diagrammatically illustrates one embodiment of a prosthesis delivery system in accordance with the invention.

FIG. 2 diagrammatically illustrates an electromagnetic field generating system for use with the prosthesis delivery system of FIG. 1.

FIG. 3 is a partial sectional view of a distal portion of the prosthesis delivery system of FIG. 1 coupled to the circuit of FIG. 2.

FIG. 4 schematically illustrates one embodiment of a multiple coil sensor which can be used in the various embodiments described herein.

FIG. 5 is an end view of the prosthesis delivery system of FIG. 1 taken from 5-5 in FIG. 1 showing optional sensors and accompanying carrier arms.

FIGS. 6-15 illustrate a method of stent-graft deployment in accordance with the invention, where FIGS. 6, 7, 8, and 9 illustrate advancing the prosthesis delivery system of FIG. 1 from a femoral artery to the vicinity of a renal artery; FIG. 10 depicts sensor movement into a renal artery indicating renal artery location; FIG. 11 depicts renal artery location confirmation; FIG. 12 depicts stent-graft deployment adjacent to the located renal artery; FIG. 13 depicts obtaining a position in three dimensions in the contralateral stent-graft short leg using a sensor; FIG. 14 illustrates cannulating the contralateral stent-graft short leg with a contralateral catheter having a sensor attached to a distal portion thereof; and FIG. 15 illustrates the full deployment of the modular bifurcated stent-graft of FIG. 14 with an optional distal bare spring wire.

FIGS. 16 and 17 are flow charts for the method of FIGS. 6-15.

FIG. 18 diagrammatically illustrates another embodiment of a prosthesis delivery system in accordance with the invention.

FIG. 19 provides a schematic sectional view to help illustrate a method of using the prosthesis delivery system of FIG. 18.

FIG. 20A illustrates a locating and/or mapping embodiment according to the invention in an unrestrained or free state.

FIG. 20B illustrates the locating and/or mapping embodiment of FIG. 21A in a collapsed free state.

FIG. 21 is an end view of the embodiment of FIG. 20.

FIG. 22 diagrammatically illustrates a feeler arm for use with the embodiment of FIG. 20.

FIG. 23 diagrammatically illustrates another feeler arm for use with the embodiment of FIG. 20.

FIG. 24A diagrammatically illustrates another feeler arm for use with the apparatus of FIG. 20A.

FIG. 24B illustrates a variation of the embodiment depicted in FIG. 24A

FIG. 25 diagrammatically illustrates another locating and/or mapping embodiment according to the invention.

FIG. 26 is a sectional view of the embodiment of FIG.25 taken along line 26.

FIG. 27 diagrammatically illustrates the embodiment of FIG. 25 in a closed position.

FIG. 28 diagrammatically illustrates another locating and/or mapping embodiment according to the invention.

FIG. 29 diagrammatically illustrates the embodiment of FIG. 28 in an expanded state.

FIG. 30 illustrates operation of apparatus according to one embodiment of the invention to map and locate features of vasculature.

### DETAILED DESCRIPTION

The following description will be made with reference to the drawings where when referring to the various figures, it should be understood that like numerals or characters indicate like elements.

Regarding proximal and distal positions, the proximal end of the prosthesis (e.g., stent-graft) is the end closest to the heart (by way of blood flow) whereas the distal end is the end farthest away from the heart during deployment. In contrast, the distal end of the catheter is usually identified as the end that is farthest from the operator, while the proximal end of the catheter is the end nearest the operator. Therefore, the prosthesis (e.g., stent-graft) and delivery system proximal and distal descriptions may be consistent or opposite to one another depending on prosthesis (e.g., stent-graft) location in relation to the catheter delivery path.

Embodiments according to the invention facilitate mapping of one or more branch lumens in a patient prior to stent-graft deployment and/or locating a prosthesis lumen position prior to cannulation thereof. Branch lumens emanate from the intersection of a vessel (e.g., the aorta) and other attendant vessels (e.g., major arteries such as the renal, brachiocephalic, subclavian and carotid arteries). According to one embodiment of the invention, one or more sensors, which can be signal devices (e.g., magnetically sensitive, electrically conductive sensing coils, which can be referred to as antenna coils), are coupled to a prosthesis delivery catheter through a flexible member that allows the signal device(s) to move relative to the catheter.

In the case of magnetically sensitive, electrically conductive sensing coils, the coil positions can be located by determining the positions of the coils relative to a plurality of magnetic field sources of known location. Pre-specified electromagnetic fields are projected to the portion of the anatomical structure of interest (e.g., that portion that includes all prospective locations of the coils in a manner and sufficient to induce voltage signals in the coil(s). Electrical measurements of the voltage signals are made to compute the angular orientation and positional coordinates of the sensing coil(s) and hence the location of the vasculature and/or devices of interest. An example of sensing coils for determining the location of a catheter or endoscopic probe inserted into a selected body cavity of a patient undergoing surgery in response to prespecified electromagnetic fields is disclosed in U.S. Patent No. 5,592,939 to Martinelli, the disclosure of which is hereby incorporated herein by reference in its entirety. Another example of methods and apparatus for locating the position in three dimensions of a sensor comprising a sensing coil by generating magnetic fields which are detected at the sensor is disclosed in U.S. Pat. No. 5,913,820 to Bladen, et al., the disclosure of which is hereby incorporated herein by reference in its entirety.

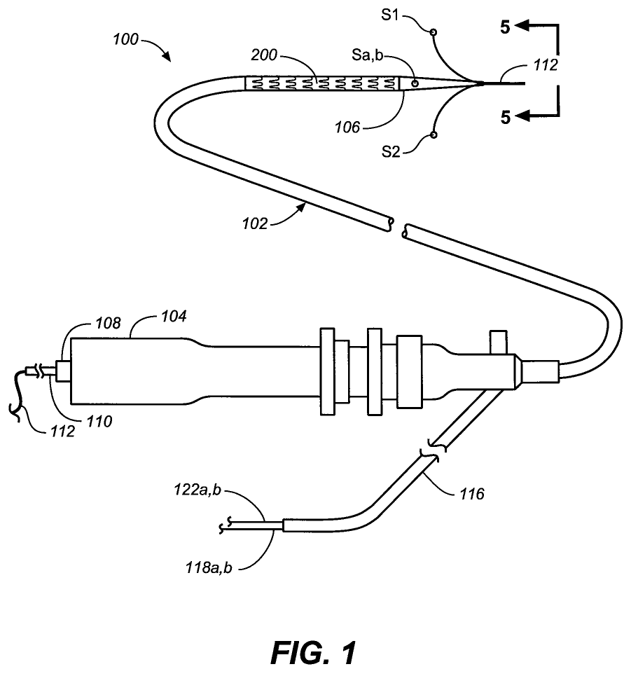

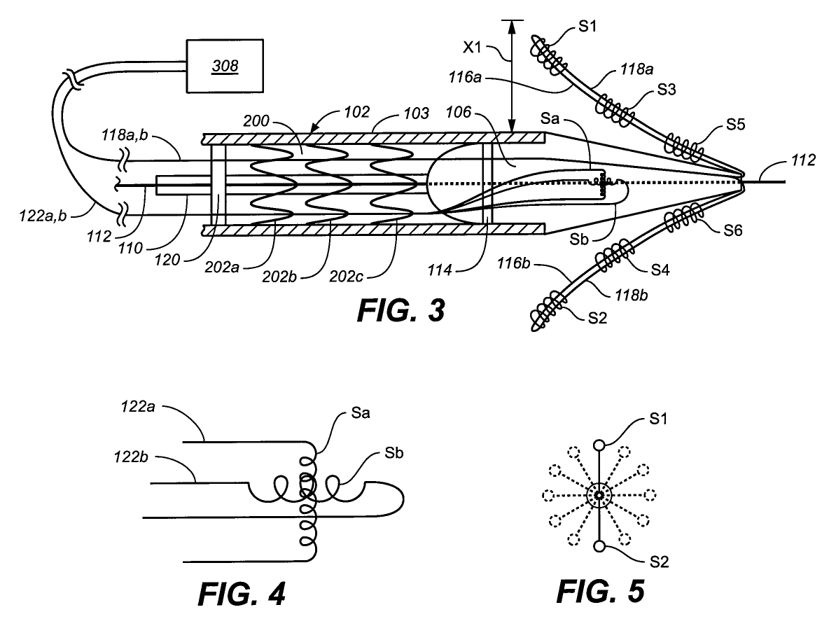

Referring to FIG. 1, a first embodiment of a prosthesis delivery system according to the invention is shown and generally designated with reference numeral 100. Prosthesis delivery system 100 comprises catheter 102, control handle 104, tapered tip member (or obturator) 106, which can form a portion of the distal end of the catheter. The tapered tip can be referred to as a probe as can the catheter-tapered tip combination. Handle 104 includes an inlet 108, through which central (inner) guidewire lumen 110 enters the handle and extends to flexible tapered tip 106, which has an axial bore for slidably receiving guidewire 112. Tapered tip member 106 is at the distal end of catheter sheath 103 (FIG. 3) and handle 104 is at to the proximal end of the catheter sheath. Guidewire 112 can be slidably disposed in guidewire lumen 110 and catheter 102 tracked thereover.

One or more markers or sensors (S1, S2 . . . Sn) are suspended from tapered tip 106. Further, one or more markers or sensors (Sa, Sb . . . Sn) are coupled to the tapered tip and can be secured to or embedded in the tapered tip as will be described in more detail below. Alternatively, sensors (Sa, Sb . . . Sn) can be coupled to the catheter sheath or guidewire lumen along the distal portion of the catheter sheath adjacent to the tapered tip.

When the prosthesis to be delivered is a self-expanding graft or stent-graft (such as stent-graft 200 shown in FIG. 3, it generally is radially compressed or folded and placed in the distal end portion of the delivery catheter and allowed to expand upon deployment from the catheter at the target site as will be described in detail below. Stent-graft 200 can include a plurality of undulating stent elements 202a,b,c to support the tubular graft material as is known in the art.

Referring to FIG. 3, catheter tube or sheath 103 (outer tube) and inner guidewire tube 110 are coaxial and arranged for relative axial movement there between. The prosthesis (e.g., stent-graft 200) is positioned within the distal end of outer tube 103 and in front of pusher member or stop 120, which is concentric with and secured to inner guidewire tube 110 and can have a disk or ring shaped configuration with a central access bore to provide access for guidewire tube 110. A radiopaque ring 114 can be provided on the proximal end of tapered tip 106 or the inside of sheath 103 to assist with imaging the tapered tip or distal end of sheath 103 using fluoroscopic techniques. Once the catheter is positioned for deployment of the prosthesis at the desired site, the inner member or guidewire lumen 110 with stop 120 are held stationary and the outer tube or sheath 103 withdrawn so that sheath 103 is displaced from tapered tip 106 and the stent-graft gradually exposed and allowed to expand. Stop 120 therefore is sized to engage the distal end of the stent-graft as the stent-graft is deployed. The proximal ends of the sheath 103 and inner tube or guidewire lumen 110 are coupled to and manipulated by handle 104. Tapered tip 106 optionally can include a stent graft proximal end holding mechanism to receive and hold the proximal end of the stent-graft so that the operator can allow expansion of the stent-graft proximal end during the last phase of its deployment. In this regard, any of the stent-graft deployment systems described in U.S. Patent Application Publication No. 2004/0093063, which published on May 13, 2004 to Wright et al. and is entitled Controlled Deployment Delivery System, the disclosure of which is hereby incorporated herein by reference in its entirety, can be incorporated into stent-graft delivery system 100.

In the embodiment shown in FIG. 3, a plurality of sensors are coupled to the catheter and suspended therefrom through flexible member 116a and a plurality of sensors are coupled to the catheter and suspended therefrom through flexible member 116b. Flexible members 116a and 116b, which can be wires, allow the sensors attached thereto to move toward or away from the catheter. Sensors S1, S3 and S5 are axially spaced from one another along flexible member 116a (with S1 at the feeler end of the flexible member) and electrically coupled to processor or measuring unit 308 through conductor or copper wire 11 8a, which can extend through the distal opening of tapered tip 106 and through guidewire lumen 110 before branching out to processor or measuring unit 308 in the vicinity of handle 104. Similarly, sensors S2, S4 and S6 are axially spaced from one another along flexible member 116a (with S2 at the feeler end of the flexible member) and electrically coupled to processor or measuring unit 308 through conductor or copper wire 118b, which can extend through the distal opening of tapered tip 106 and through guidewire lumen 110 before branching out to processor or measuring unit in the vicinity of handle 104. Each conductor or copper wire can be wound around a respective flexible member to secure the conductor and hence the sensors thereto. Each flexible member has a fixed end and a feeler end and each fixed end is attached to the distal end of tapered tip 106. In this manner, the flexible members can be used as feeler wires to find and position branch vessels such as the renal arteries.

Although the flexible members are each shown with three sensors, the number of sensors can vary. For example, a single sensor can be provided at each flexible member feeler end. However, three sensors suspended along a respective flexible member as shown in FIG. 3, provides a sufficient number of data points to provide a virtual image of the flexible member and, thus, provide a virtual image of the contour, orientation and/or direction of the branch vessel to determine, for example, if a branch vessel extends about 90 degrees or about 30 degrees from the vessel from which it branches.

In the illustrative embodiment of FIG. 4, a pair of sensors Sa and Sb are secured to the tapered tip to provide a reference signal. They can be embedded in or otherwise attached to tapered tip 106 and extended through guidewire lumen 110 and then coupled to processor or measuring unit 308. In an alternative embodiment, sensors Sa and Sb (not shown) can be secured to a distal portion of catheter sheath 103 or guidewire lumen 110. Further and as shown in the embodiment illustrated in FIG. 4, sensors Sa and Sb can be coils that are oriented perpendicular to one to another. Similar perpendicular sensor pairs can be used in place of one or more of sensors S1-S6 shown in FIG. 3.

Referring to FIG. 5, where optional flexible members are shown in dashed line, it is to be understood that the number of flexible members having one or more sensors coupled or secured thereto or suspended thereby can vary. Further, a single flexible member with one or more sensors coupled or secured thereto can be used.

Each flexible member 116a and 116b can be made from shape memory material and provided with a preshaped memory set configuration such as the configuration shown in FIG. 3. For example, flexible members 116a and 116b can be nitinol wire and can be placed in the desired shape (e.g., that shown in FIG. 3) and heated for about 5-15 minutes in a hot salt bath or sand having a temperature of about 480-515° C. They can then be air cooled or placed in an oil bath or water quenched depending on the desired properties. In one alternative, flexible members 116a and 116b can be stainless steel and preshaped with known techniques to assume the configuration shown in FIG. 3.

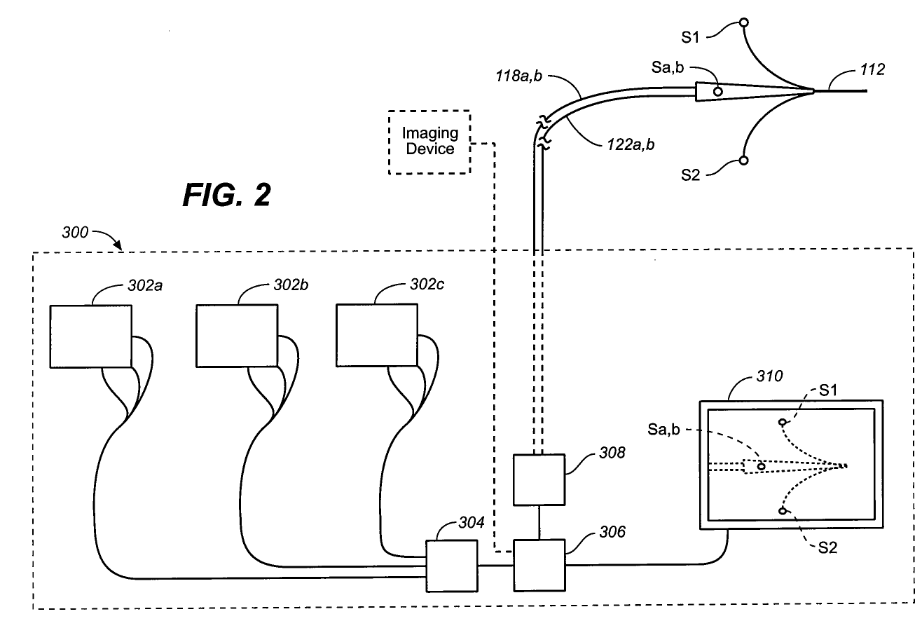

Any suitable electromagnetic field generating and signal processing circuit for locating sensor position in three dimensions can be used (see e.g., U.S. Pat. No. 5,913,820 to Bladen, et al. (supra) regarding magnetically sensitive, electrically conductive sensing coils (e.g., antenna coils)). Referring to FIG. 2, one such field generating and signal processing circuit configuration for generating magnetic fields at the location of the sensors and processing the voltage signals that the sensors generate in response to the generated magnetic fields, when the sensors are conductive sensing coils, is generally designated with reference numeral 300.

Circuit 300 generally includes three electromagnetic field (EMF) generators 302a, 302b, and 302c, amplifier 304, controller 306, measurement unit 308, and display device 310. Each field generator comprises three electrically separate coils of wire (generating coils), which can be wound about a cuboid wooden former. The nine generating coils are separately electrically connected to amplifier 304, which is able, under the direction of controller 306, to drive each coil individually.

In use, controller 306 directs amplifier 304 to drive each of the nine generating coils sequentially. Once the quasi-static field from a particular generating coil is established, the value of the voltage induced in each sensing coil (S1-S6) by this field is measured by the measurement unit 308, processed and passed to controller 306, which stores the value and then instructs the amplifier 304 to stop driving the present generating coil and to start driving the next generating coil. When all generating coils have been driven, or energized, and the corresponding nine voltages induced into each sensing coil have been measured and stored, controller or processor 306 calculates the location and orientation of each sensor relative to the field generators and displays this on a display device 310. This calculation can be carried out while the subsequent set of nine measurements is being taken. Thus, by sequentially driving each of the nine generating coils, arranged in three groups of three mutually orthogonal coils, the location and orientation of each sensing coil can be determined.

The sensor and generating coil specifications, as well as the processing steps are within the skill of one of ordinary skill of the art. Examples of coil specifications and general processing steps that can be used are disclosed in U.S. Pat. No. 5,913,820 to Bladen, et al., the disclosure of which is hereby incorporated herein by reference in its entirety.

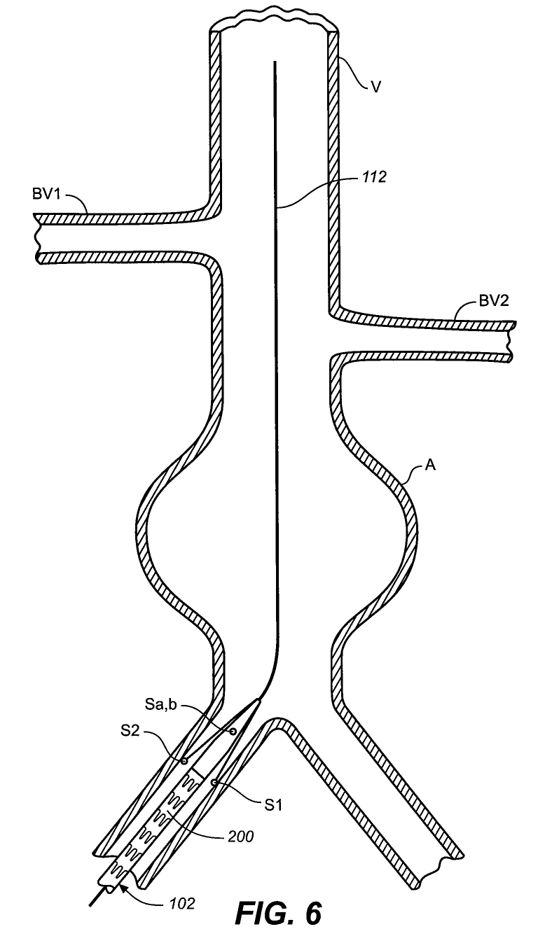

Referring to FIGS. 6-15, an exemplary operation of the system will now be described. For the purposes of the example, the procedure involves the endovascular delivery and deployment of an abdominal aortic aneurysm (AAA) bifurcated stent-graft.

Prior to the surgical procedure, the patient is scanned using either a CT or MRI scanner to generate a three-dimensional model of the vasculature to be tracked. Therefore, the aorta and branch vessels of interest (e.g., renal arteries) can be scanned and images taken there along to create a three-dimensional pre-procedural data set for that vasculature and create a virtual model upon which real-time data will be overlayed. This information is stored in the system (e.g., it can be input into controller 306 of system (circuit) 300) and is identified and accessible as a historical baseline image. Any portion of the aorta or branch vessels can be provided with fiducial markers (anatomic markers which are considered to provide a reliable reference to a particular body location) that are visible on the pre-procedural images and accurately detectable during the procedure as is known in the art. The imaging device depicted in FIG. 2 represents either a preoperative imaging device as described above or an intra-operative imaging device subsequently used in the procedure.

The three magnetic field generators are positioned on the operating table to facilitate triangulation of the exact position of each sensor in three-dimensional space using xyz coordinates.

The patient is prepared for surgery and a cut is made down to a femoral artery and a guidewire (by itself or together with a guide catheter) inserted. A contrast agent catheter is delivered through the femoral artery and the vasculature perfused with contrast and a fluoroscopic image including the renal arteries taken. Using the fiducial markers, the processor orients or registers the previously acquired and stored three-dimensional image to the currently presented fluoroscopic X-ray image.

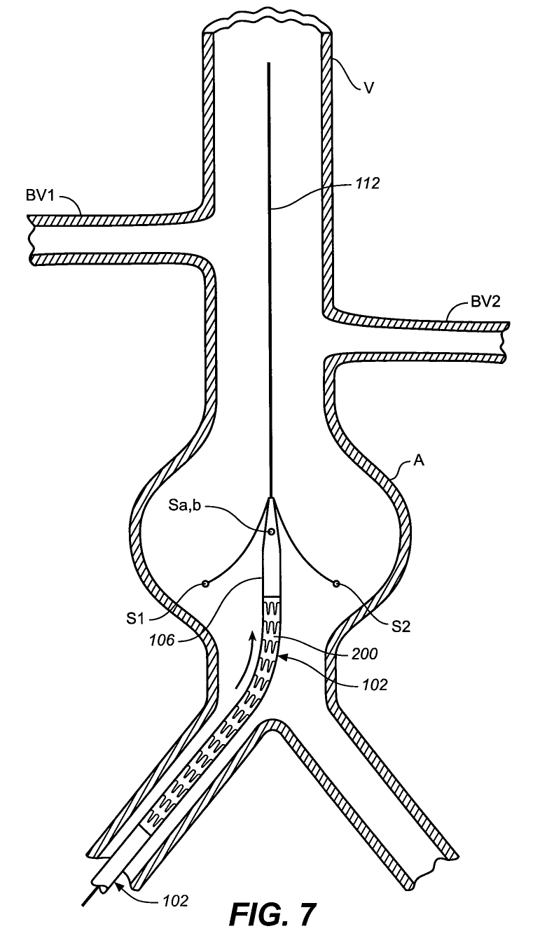

Referring to FIG. 6, the operator tracks catheter 102 over guidewire 112 toward aneurysm A and branch vessels BV1 and BV2, which branch from vessel V, which in this example is the aorta. The position of the distal end of the catheter is monitored virtually based on the known catheter dimensions entered into the processor and the signals from sensors (or coils) S1, S2, Sa and Sb, which identify their position in the three-dimensional model. The display will show the position of the sensors, which may be referred to as markers, tracking the profile of the vessel wall. The operator may visualize the displacement of sensors S1 and S2 as the tapered tip passes through aneurysm A (FIG. 7), where the walls of the aneurysm bulge so much (may be extended (distended) to the extent) that they do not contact or constrain flexible members 116a,b. The flexible members (or feeler wires) 116a,b are then free to move toward or to their undeformed free state (memory set configuration). In this state, end sensors S1 or S2 can be radially spaced a distance X1 (FIG. 3) measured from the juncture of the catheter and tapered tip in an orthogonal direction extending radially outward therefrom. X1 typically is about 18 mm to 36 mm, but can vary according to the application.

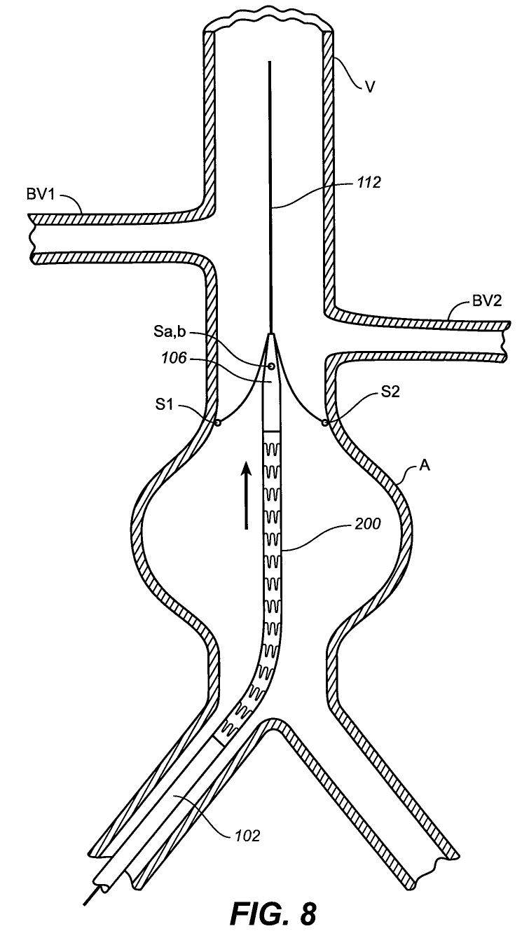

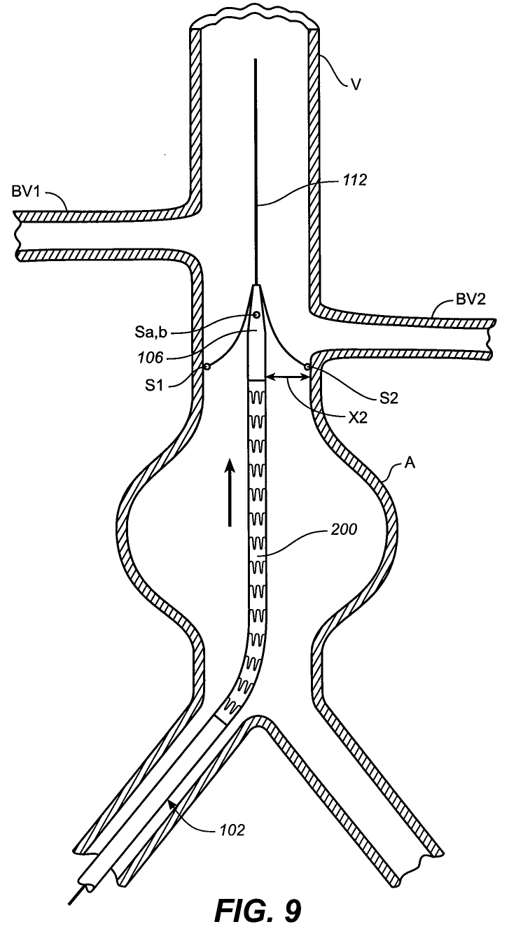

The catheter is further advanced to where the sensors reach the aneurysm's proximal neck as shown in FIG. 8 where they move radially inward toward catheter tapered tip 106. Their position continues to be relayed to the operator as they move along the proximal neck to a point where they are radially spaced from the catheter a distance X2 (measured from the juncture of the catheter and tapered tip and in an orthogonal direction extending radially outward therefrom) as shown in FIG. 9.

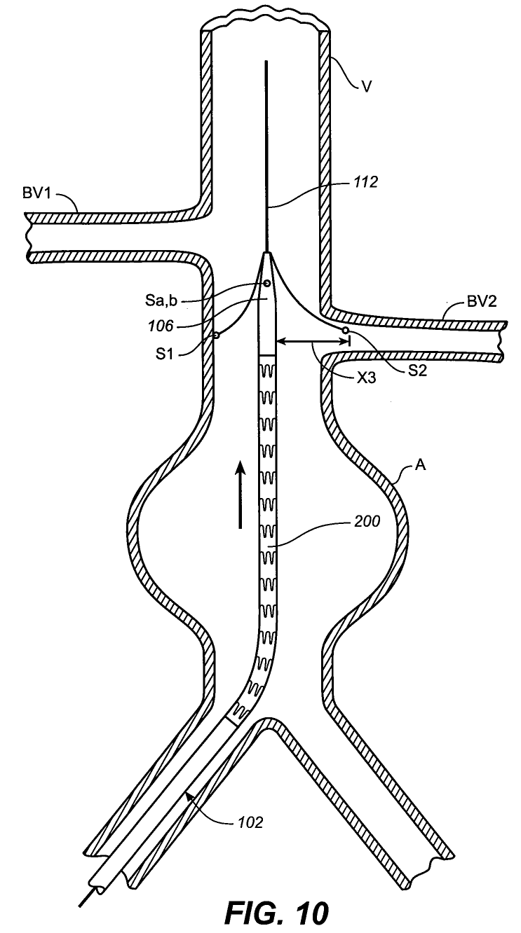

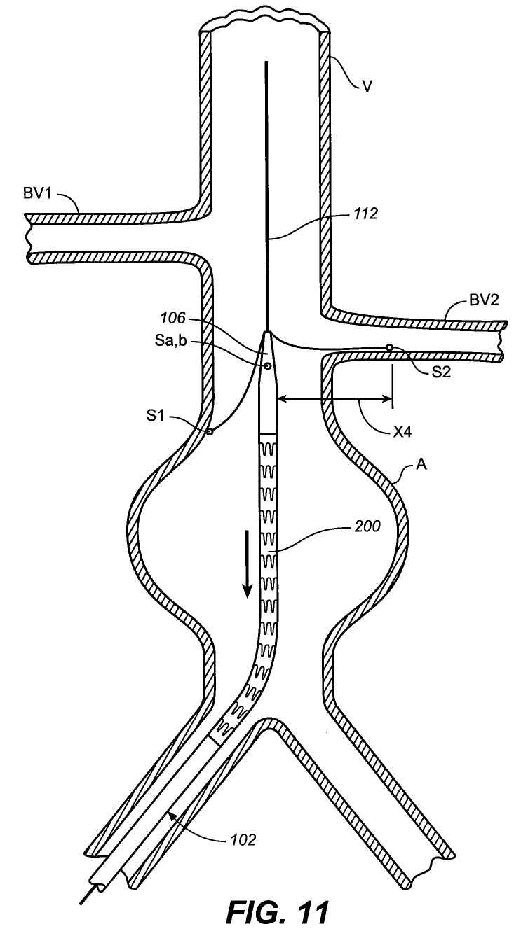

In the vicinity of the target location (e.g., the lower renal artery), which the operator can estimate based on the three-dimensional model and the sensor positions, the operator rotates and further advances the catheter to find the lower renal artery, which in this example corresponds to BV2. When a sensor indicates movement in a direction radial outward from tapered tip 106 that exceeds the expected position of the vessel wall, the operator can conclude that the renal artery has been found. Referring to FIG. 10, the position of the sensor can be determined and the determined position used to calculate the distance (e.g., distance X3), between the sensor and the catheter (measured from the juncture of the catheter and tapered tip in an orthogonal direction extending radially outward therefrom) as an indicator of the sensor being located in the renal artery. Alternatively, the operator can simply qualitatively track the magnitude of sensor radial outward movement on the three-dimensional model as displayed on the monitor as an indicator of the renal artery opening location. In either case, the operator may confirm detection of the renal artery opening by slowly withdrawing the catheter to see if the sensor moves farther away from the catheter in a radial direction. One example, of such movement is shown in FIG. 11. As described above, the position of the sensor can be determined and the determined position used to calculate its distance (e.g, distance X4 between the sensor and the catheter measured from the juncture of the catheter and tapered tip in an orthogonal direction extending radially outward therefrom).

If the aorta was very tortuous, the catheter may have significantly changed the aorta's configuration during advancement therethrough. In this event, the surgeon has the option to take a fluoroscopic image to confirm the location of the renal artery.

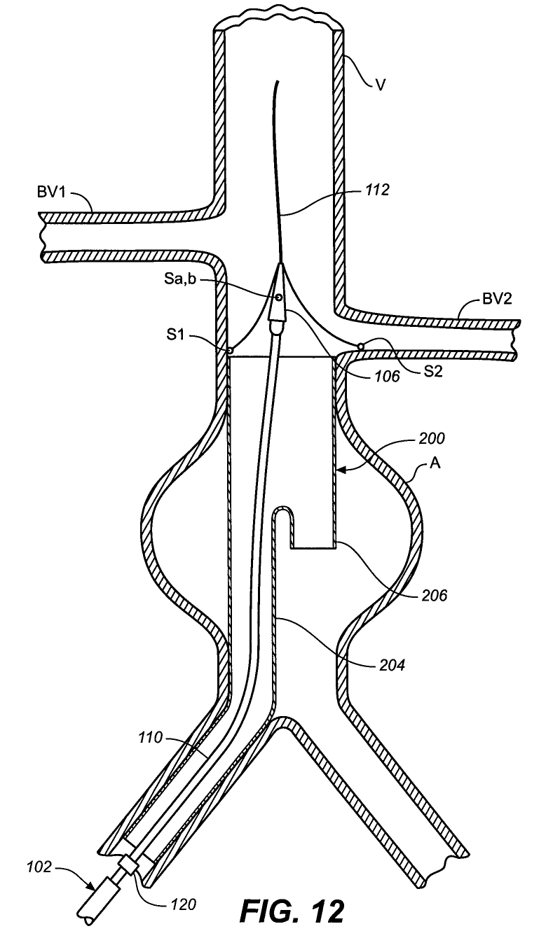

Locating the upper and lower walls of the renal artery provides a guide for the location of the ostium of the renal artery and is related to fiducial markers already present in the anatomy, the stent-graft is positioned at the desired location relative to the three-dimensional model. Since the position of the proximal end of stent-graft 200 relative to sensors Sa,b is known, the proximal end of the stent-graft can be positioned at the desired location relative to the renal artery. The catheter is advanced to align sensors Sa,b with S2, while monitoring these sensors on the display, and then advanced a distance slightly less than the distance between sensors Sa,b and the stent-graft to align the stent-graft with the proximal neck landing zone. Alternatively, one, two or more sensors can be coupled to the catheter sheath or inner surface of guidewire lumen 110 to indicate the exact position of the proximal end of the stent-graft. Once the stent-graft is in the desired position, the operator holds the guidewire tube 110 and pusher disk 120 stationary and retracts or pulls back sheath 103 (FIG. 12).

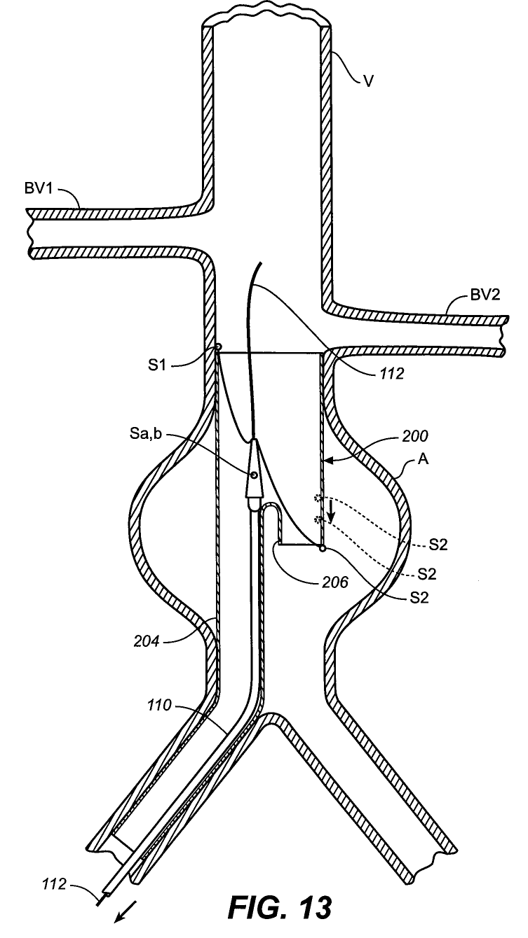

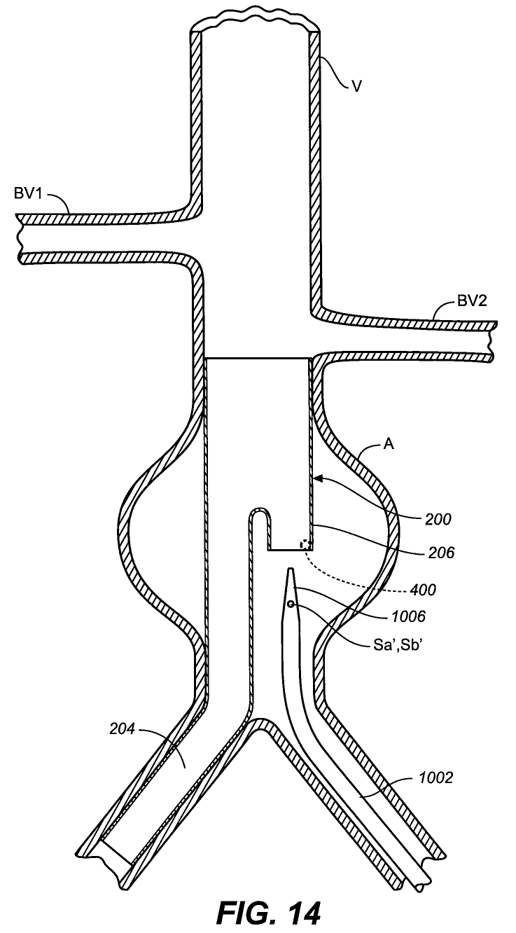

Referring to FIG. 13, the catheter is then retracted to position a sensor (e.g., S2) in the contralateral short leg of modular bifurcated stent-graft 200 as shown in FIG. 13 where the position of sensor S2 is shown in dashed line as it tracks along an inner surface of the short leg (contralateral section) until it reaches the end of the short leg from where it moves radially outward. This information allows the operator to record in memory in the three-dimensional image a target position inside the contralateral section or trunk 206 shown in dashed line and designated with reference numeral 400 (FIG. 14).

Referring to FIG. 14, a steerable catheter 1002, which can have a similar sheath, guidewire lumen and tapered tip construction as catheter 102, is similarly introduced into the contralateral femoral artery in a conventional manner. Tapered tip 1006 includes sensors Sa′ and Sb′, which can be oriented and coupled to tapered tip 1006 and constructed in the same manner as sensors Sa and Sb (not shown) are oriented and coupled to tapered tip 106. As steerable catheter 1002 is advanced, the operator uses the three-dimensional image or model to track tapered tip 1006, which leads to the opening of the short or stub leg. If sensor S2 has not been retracted, tapered tip 1006 can be guided toward sensor S2. If sensor S2 has been withdrawn, tapered tip 1006 is guided toward position 400, the acquired data for which has been stored in the processor. By either moving the sensors closer to one another, while viewing their relative positions as displayed on the monitor or guiding tapered tip 1006 toward position 400, while both are displayed on the three-dimensional model, the operator cannulates the contralateral gate of trunk 206 with catheter 1002.

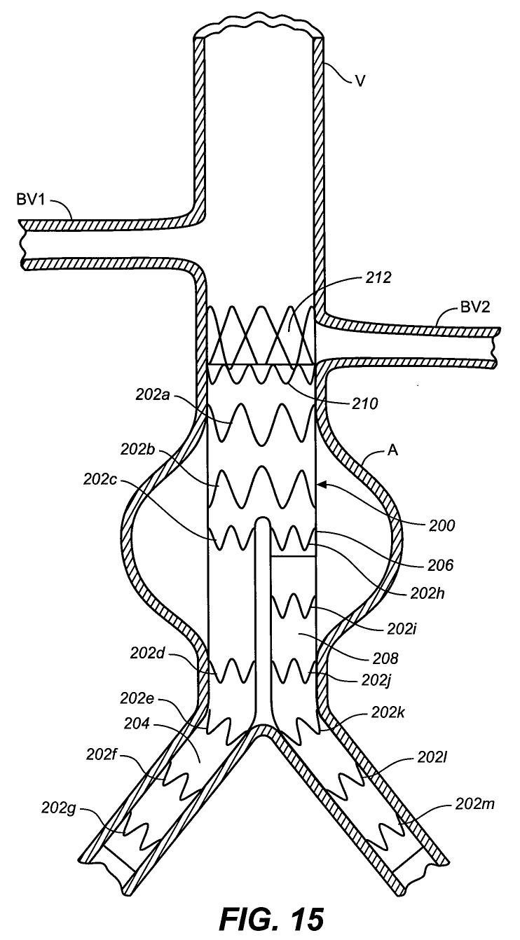

Referring to FIG. 15, the operator then deploys contralateral leg stent-graft section 208 by retracting the catheter sheath in a manner similar to deploying stent-graft 200. The deployed bifurcated stent graft can include a plurality of undulating stents 202a-m secured to the inner or outer wall of the bifurcated tubular graft material (which can comprise, for example, Dacron® or expanded polytetrafluoroethylene (ePTFE)), undulating support wire secured to the inner or outer wall of the proximal portion of the tubular graft, and bare spring 212, which can be secured to the proximal portion of the tubular graft. Bare spring 212 can be flared outwardly moving in a proximal direction to enhance stent-graft anchoring. Sutures or any other suitable means can be used to secure the stents, support wire, and bare spring to the graft material.

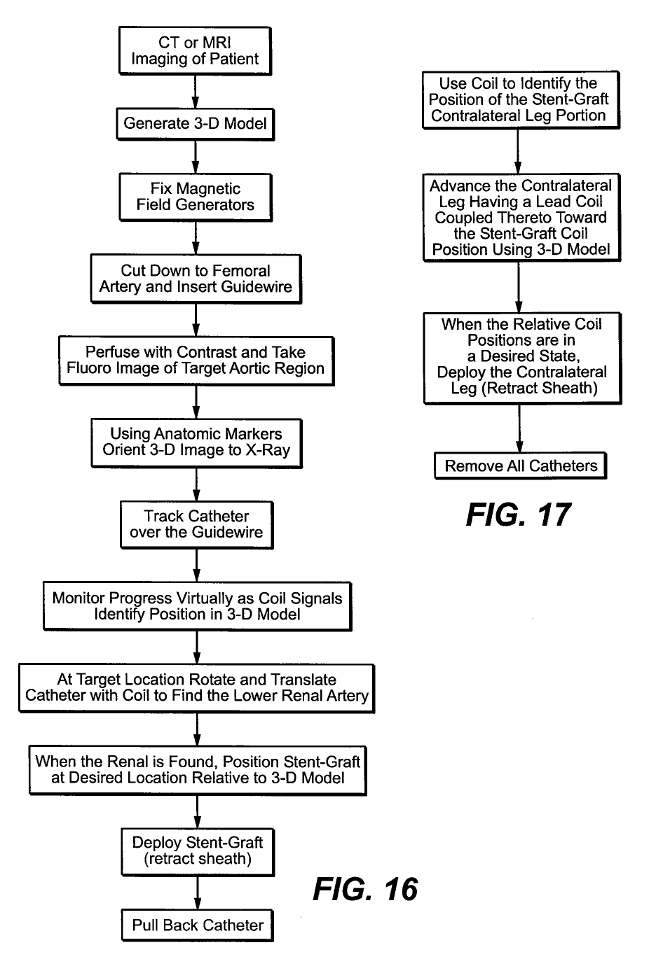

All catheters are then removed. A flow chart summary of the foregoing procedure is depicted in FIGS. 16 and 17.

The three-dimensional data points used in the procedure can increase accuracy of the surgery as compared to two-dimensional fluoroscopic images. The need for contrast agent also can be eliminated or minimized.

In another embodiment according to the invention, a self-contained proximity based system, which does not require external field generators, identifies when the distance between two or more markers or signal devices increases to indicate the position of a branch vessel such as a renal artery.

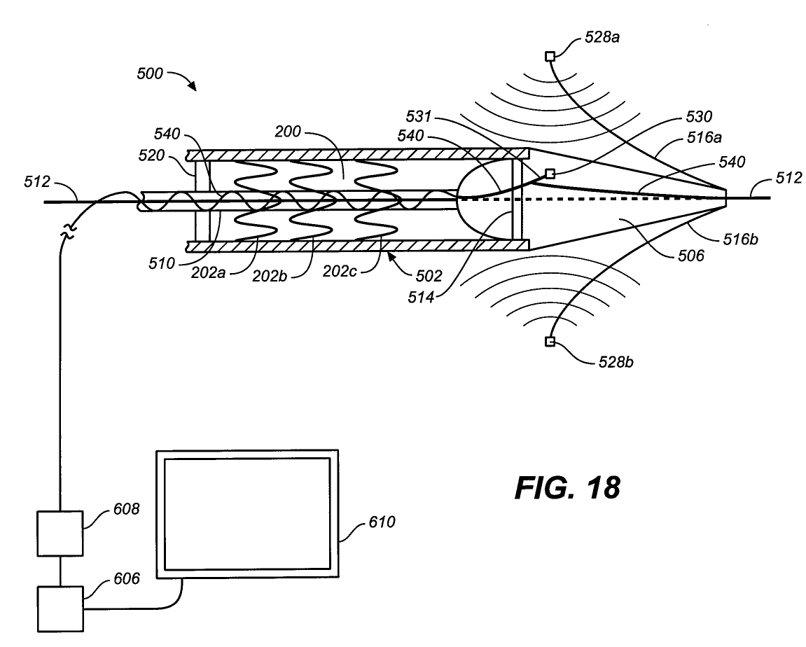

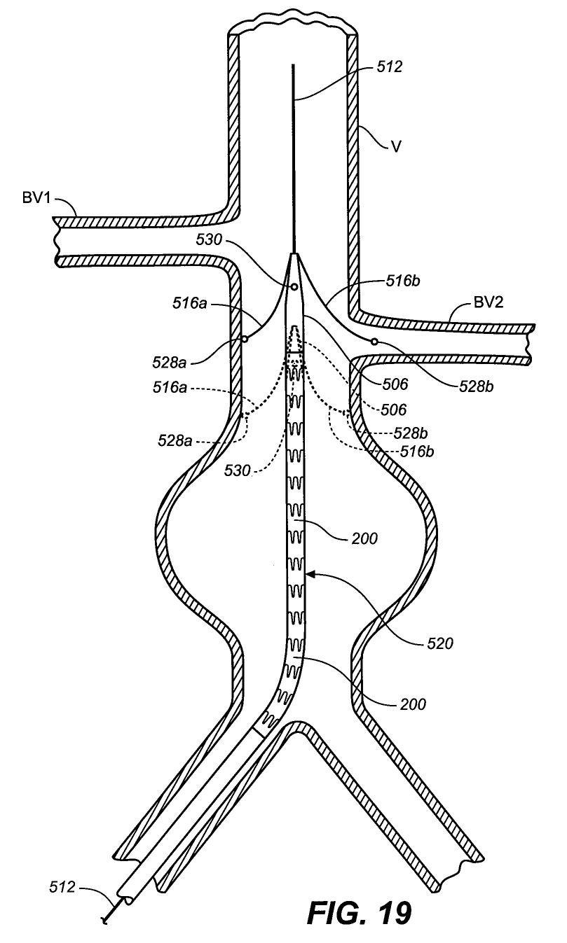

Referring to the illustrative example of FIGS. 18 and 19, stent-graft delivery system 500 includes catheter 502, control handle, tapered tip 506, guidewire lumen 510, guidewire 512, radiopaque ring 514, flexible members 516a and 516b, and pusher disk 520, which can correspond or be similar to catheter 102, control handle, tapered tip, 106 guidewire lumen 110, guidewire 112, radiopaque ring 114, flexible members 116a and 116b, and pusher disk 120. Tapered tip 506 can be referred to as a probe as can the combination of catheter 502 and tapered tip 506.

In this embodiment a signal or wave generating device or transmitter 528a is secured to the feeler end of flexible member 516a or in the vicinity thereof and a signal or wave generating device or transmitter 528b is secured to the feeler end of flexible member 516b or in the vicinity thereof. A conductor can extend from each signal transmitter along a respective flexible member to lead 540a, which extends from the distal end of the tapered tip and then is incorporated into lead bundle 540 where it extends through the guidewire lumen to a power source (not shown) to controllably actuate signal generators 528a and 528b to generate RF, infrared, or electromagnetic signals or waves.

The embodiment illustrated in FIG. 18 also includes a sensor or signal receiver 530, which is embedded or otherwise secured to tapered tip 506 or catheter 502. In an alternative embodiment, receiver 530 can be secured to the distal portion of guidewire lumen 510. Receiver 530 receives the signals from signal generators 528a and 528b and transmits them via lead 540b, which with lead 540a is bundled into lead bundle 540 which is coupled to measuring unit 608, which in turn is coupled to controller 606 and display 610. Measuring unit 608 calculates the position of the sensors relative to one another based on the difference between the received signals (e.g., the difference in intensity or quality between the signals). Controller 606 processes this information for display on display 610 as units of distance (e.g., millimeters) over time. Measuring unit 608 and processor 606 can be incorporated into a single processor or unit as would be apparent to one of ordinary skill in the art.

Referring to FIG. 18, each signal generator 528a and 528b will be at a fixed distance from sensor 530, the reference position or point, when flexible members 516a and 516b are in a relaxed, undeformed or free state (i.e., in their memory set configuration). As the catheter is advanced through vessel V past aneurysm A as shown in FIG. 19, the flexible members 516a and 516b urge the signal generators against the proximal neck or landing zone of the aneurysm. In this position, the signal generators shown in dashed line. The catheter is further advanced with optional rotation until one signal generator moves into branch vessel BV2 (e.g., a renal artery) to a second position shown in solid line. The movement is in response to the respective flexible member being allowed to move toward its memory shape when it reaches the opening in the vessel wall leading to the branch vessel. The change in the relative position of signal generator 528b and signal receiver 530 versus signal generator 528a and signal receiver 530 indicates that a branch vessel has been detected. The position of 528a to 530, and 528b to 530, and the addition of those two values would be digitally displayed on a monitor.

In a variation of system 500, signal device 530 can be a signal generator and signal devices 528a,b can be signal receivers. As in the embodiment of FIG. 3, a plurality of sensors or sensing coils can be provided on each flexible member in this variation to assist in the proximity evaluation and virtual imaging of the contour, orientation and/or direction of the branch vessel opening. The refinement of the image generally depends on the number of sensors used.

Any of the foregoing embodiments also can be used to obtain three-dimensional data indicative of the opening of branch vessels (e.g. the renal arteries) in applications where there is insufficient proximal neck to anchor the proximal end of the stent-graft. In this case, the stent-graft is positioned across one or both of the branch vessels (e.g., renal arteries) and the acquired position data used to track a steerable piercing catheter having a sensor or signal device coupled to the distal end portion thereof so that the piercing catheter can be guided through the stent-graft and into the either or both branch vessel openings. Alternatively, the stent-graft can include one or more openings, each of which have a recorded position relative to the tapered tip sensor or signal device(s) or one or more sensors attached to the guidewire lumen as described above so that the position of the stent-graft openings can be virtually tracked along the three-dimensional model that has been updated to include the opening position(s).

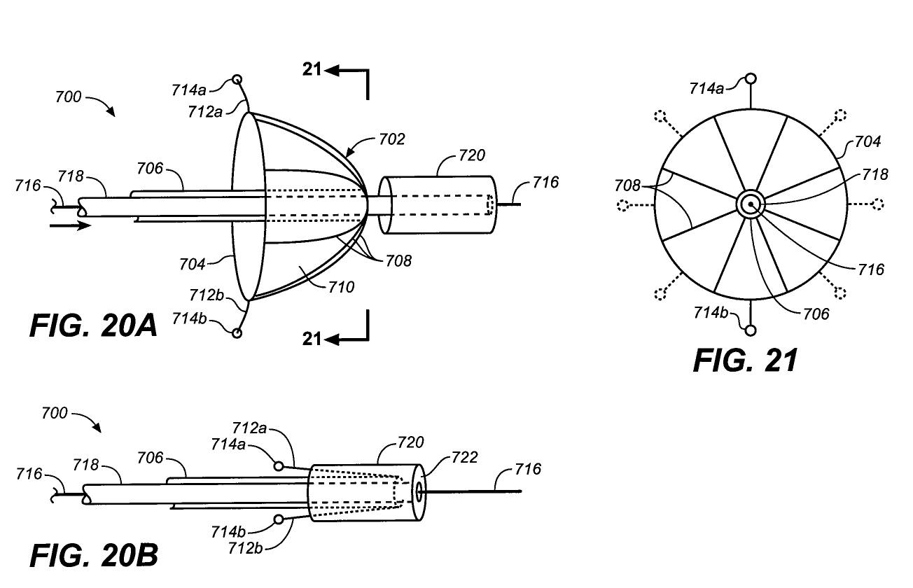

Referring to FIGS. 20A and 20B, another anatomical locator and/or mapping device is shown and generally designated with reference numeral 700. Device 700 comprises a collapsible support structure, which in the exemplary embodiment depicted in FIG. 20A, is shown with an umbrella configuration and generally designated with reference numeral 702. Umbrella 702 includes hoop or circumferential wire 704 and radial wire supports 708, which together form a collapsible canopy that is secured to the distal end portion of elongated tubular member or guidewire tube 706. Optionally, umbrella material 710 can be secured to the umbrella structure so as to cover radial wire supports 708 and form a generally continuous canopy. Typically, such umbrella material 710 will be selected to comprise material that allows blood to perfuse through the material. Tubular member 706 can be concentrically positioned within wire supports 708 as shown in FIG. 20A. One end of each of wire support 708 is secured to tubular member 706 with any suitable means such as glue or solder. The other ends of supports 708 are secured to hoop or circumferential wire 704 by any suitable means, which also can be glue or solder.

A plurality of markers or sensors 714a, 714b, . . . 714n are coupled to umbrella 702 through radially extending flexible support members or feeler arms 712a, 712b, . . . 712n. In the illustrative example, each of feeler or support arms 712a and 712b has one end secured to hoop or circumferential wire 704 and its other end secured to a respective marker or sensor 714a and 714b so that the markers or sensors are radially spaced from the hoop or circumferential wire 704 as well as tubular member 706. Although two markers or sensors are shown in FIGS. 20A and 20B, additional markers or sensors can be used as shown in phantom in FIG. 21. Typically, about two to eight markers or sensors are used, but more or fewer markers or sensors can be used as well. Generally speaking, more markers cover more vessel surface area and improve the resolution of the image thereof. When a single marker is used, it typically will be advanced along the vessel wall in both a circumferential and axial direction and, for example, can be tracked along a spiral or helical path to cover more surface area than a single axial pass would. Alternatively, the operator can make multiple passes along the target area with the single marker or this can be done in combination with circumferential and axial tracking. When a plurality of markers are selected, typically at least four will be used. In this case, the sensors also can be moved in a circumferential direction while being advanced up the vessel as well and/or can be passed along the target area a plurality of times to acquire more data relating to the inner wall surface of the vessel. Typically, the plurality of markers will be equidistantly spaced in a circumferential direction about hoop or circumferential wire 704 as shown in FIG. 21. However, it should be understood that other configurations or arrangements can be used.

Tubular member 706 is sized so that it can pass over guidewire 716 so that anatomical locator and/or mapping device 700 can be delivered to the desired site. In this manner, the markers or sensors (e.g., markers or sensors 714a and 714b) are coupled to the guidewire 716 through hoop 704, radial support arms 708, and tubular member 706.

The diameter of the device from marker or sensor 714a to marker or sensor 714b and the length of the umbrella measured along the longitudinal axis of tubular member 706 can vary depending on the application. In aortic applications, this dimension typically can range from about 2.5 cm to 5 cm and the length of the umbrella 702 can be about 2 cm. The hoop or circumferential wire 704, axial wire supports 708, and sensor support arms 712a,b can be comprise any suitable material such as nitinol wire (e.g., 0.01 inch diameter nitinol wire). Further, the feeler or support arms 712a,b are constructed with the desired flexibility so as minimize or eliminate trauma resulting from contact between the markers or sensors and the anatomical surface being tracked such as the inner wall surface of an aortic aneurysm. They can have a constant flexibility, varying flexibility, or sections having different flexibilities as described in more detail below.

Device 700 optionally can include restraining apparatus to restrain umbrella 702 in a collapsed state as shown in FIG. 20B. The restraining apparatus in the illustrative example comprises a tubular member or restraint 718, which is sized to be slidably movable in tubular member 706 and to allow passage of guidewire 716 therethrough, and tubular member 720. Tubular member or restraint 720 has an open proximal end for passing over umbrella 702 and a distal end having an annular wall 722 with an opening. The distal end of tubular member or restraint 718 is secured to annular wall 722 with any suitable means such as gluing and arranged so that the lumen of tubular member or restraint 718 is aligned with the opening in annular wall 722 to allow guidewire 716 to pass through annular wall 722. Moving tubular members 706 and 718 relative to one another a sufficient distance allows one to expand or collapse umbrella 702 to assist with delivery to a target area or withdrawal therefrom. For example, when tubular member 706 is held stationary and tubular member or restraint 718 advanced proximally, restraining apparatus tube or cylinder 720 slides away from umbrella 702 and allows umbrella 702 to expand as shown in FIG. 20A. When, however, tubular member 706 is held stationary and tubular member 718 retracted or moved proximally, tube or cylinder 720 moves over umbrella 702 collapses the umbrella to return the it to a collapsed state as shown in FIG. 20B. The proximal ends of concentrically oriented tubular members 706 and 718 can be secured to a holding device of any suitable construction to allow the operator to move the tubes relative to one another.

Referring to FIG. 22, one variation of the feeler or support arms is shown and generally designated with reference numeral 722. Feeler or support arm 722 has one end secured to marker or sensor 714 with any suitable means and another end secured to hoop 704 with any suitable means as described above. Feeler or support arm 722 is tapered with a decreasing cross-section in the axial direction to provide increasing flexibility in the direction toward marker or sensor 714. Support arm 722 can be conically shaped or have another configuration which tapers toward the end to which the marker or sensor is secured.

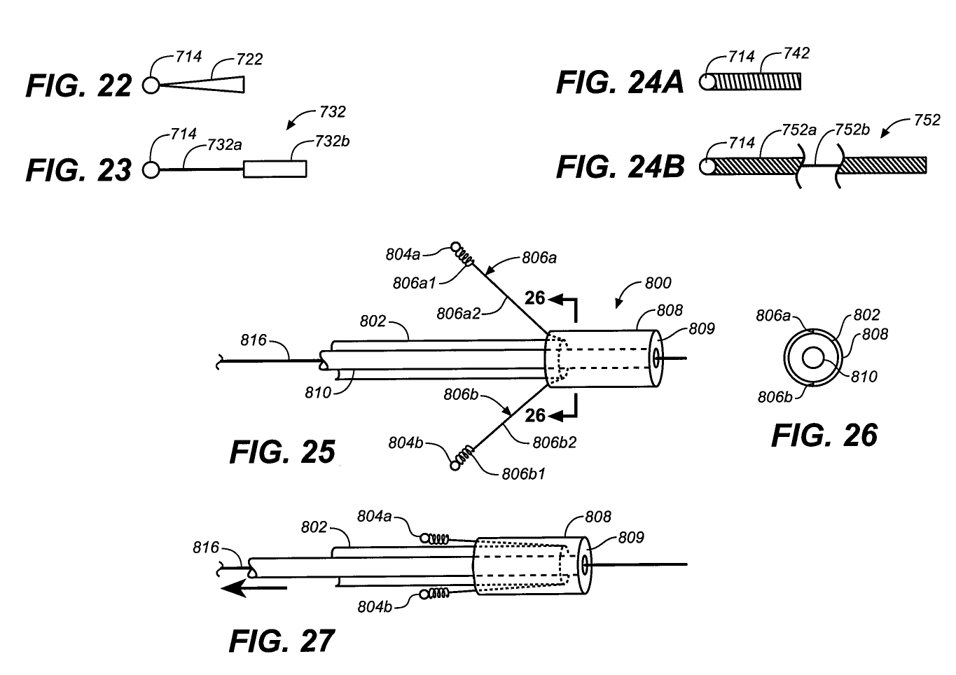

Referring to FIG. 23, another variation of the feeler or support arms is shown and generally designated with reference numeral 732. Feeler or support arm 732 has a first section 732a of constant transverse cross section and second section 732b of constant transverse cross section where the area of a transverse cross section of section 732a is less than that of section 732b so that section 732a is more flexible than section 732b. Feeler or support arm 732 has one end secured to marker or sensor 714 with any suitable means and its other end secured to hoop 704 with any suitable means as described above.

Referring to FIG. 24A, another variation of the feeler or support arms is shown and generally designated with reference numeral 742. Feeler or support arm 742 has one end secured to marker or sensor 714 with any suitable means as and its other end secured to hoop 704 with any suitable means as described above. In this illustrative example, feeler or support arm 742 comprises a wire coil having a constant pitch, which provides a substantially constant flexibility there along. Alternatively, and as shown in FIG. 24B and generally designated with reference numeral 752, the feeler arm can be comprise a wire coil having a pitch less steep than that illustrated in connection with feeler arm 742 and further include an internal wire 752b, which coil 752a surrounds and which has one end secured to hoop 704 and its other end secured to marker or sensor 714 with any suitable means as described above.

Referring to FIGS. 25-27, another anatomical locator and/or mapping device is shown and generally designated with reference numeral 800. In the illustrative embodiment, locator and/or mapping device 800 includes support member or tube 802, markers or sensors 804a and 804b, and flexible members or feeler arms 806a and 806b, which have one end secured to the distal end or end portion of support or tubular member 802 and a free end or end portion secured to a respective sensor 804a,b.

Restraining apparatus also is provided in device 800 and comprises tubular member or restraint 808 and tubular member or guidewire tube 810, which is sized to be slidably movable in tubular member 802 and to allow passage of guidewire 816 therethrough. Tubular member or restraint 808 has an open proximal end for passing over feeler arms 806a,b and a distal end having an annular wall 809 with an opening for allowing guidewire 816 to pass therethrough. The distal end of tubular member 810 is secured to annular wall 809 with any suitable means such a gluing and arranged so that the lumen of tubular member 810 is aligned with the opening in annular wall 809 to allow guidewire 816 to pass therethrough. Moving tubular members 802 and 810 relative to one another a sufficient distance allows one to permit the feeler members to radially expand (FIG. 25) or to move the feeler members radially inward to a radially collapsed or compressed state (FIG. 27) to assist with delivery to a target area or withdrawal from a vessel. For example, when tubular member 802 is held stationary and tubular member or guidewire tube 810 is advanced distally, tubular restraint 808 slides away from the feeler arms and allows them to expand as shown in FIG. 25. When, however, tubular member 802 is held stationary and tubular member or guidewire tube 810 retracted or moved proximally, tube or cylinder 808 moves over feeler arms 806a,b and collapses them as shown in FIG. 27.

Each of feeler arms 806a,b has a relatively rigid or stiff section 806a2 and 806b2 and a relatively flexible section 806a1 and 806a2 (e.g., section 806a1 is more flexible than section 806a2). In the illustrative example, relatively rigid sections 806a2,b2 comprises a wire member and relatively flexible sections 806a1,a2 comprise coils or springs to which sensors 804a and 804b are fixedly attached. The flexible sections minimize or eliminate traumatic contact between a respective sensor and the vasculature which it contacts, while the relatively rigid or stiff section is provided with a memory configuration as shown in FIG. 25 to which it tends to move when unrestrained by tubular restraint 808. This can be accomplished by attaching each feeler member to tubular member 802 so that it extends radially outward as shown FIG. 25. In this manner, when restraint 808 is sufficiently advanced, the feeler arms move away from the position shown in FIG. 27 toward the configuration shown in FIG. 25.

The proximal ends of concentrically oriented tubular members 802 and 810 can be secured to a holding device of any suitable construction to allow the operator to move the tubes relative to one another. In this manner, tubular member 802 and tubular member 810 can be moved relative to one another so that tubular restraint 808 is advanced distally and away from the proximal end of the apparatus to uncover feeler arms 806a,b and allow the feeler arms to radially expand and move toward their preshaped configuration as shown in FIG. 25. Alternatively, tubular restraint 808 can be slid over tubular member 802 and feeler arms 806a,b to radially compress the feeler arms such that they are generally parallel with the longitudinal axis of tubular member 802 and guidewire 816 as shown in FIG. 27.

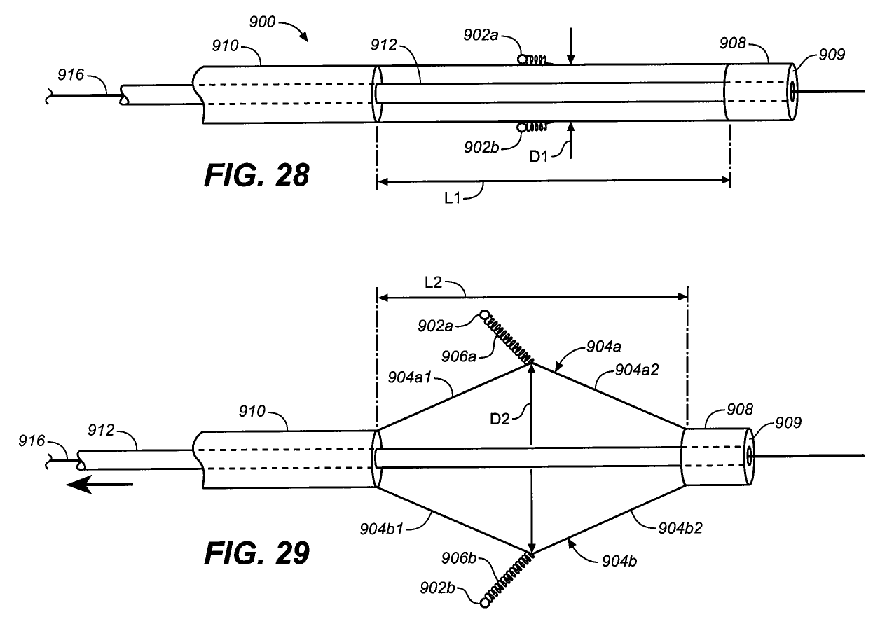

Referring to FIGS. 28 and 29, another anatomical locator and/or mapping device is shown and generally designated with reference numeral 900. Locator and/or mapping device 900 includes sensors 902a and 902b and a support structure comprising sensor support structure members 904a,b and sensor support or feeler arms 906a,b. Support structure member 904a can comprise wire and includes proximal section 904a1 and distal section 904a2, which can be made separately and subsequently joined or they can be integrally formed as a single support member 904a. The distal end of support member 904a is secured to tubular end member 908 and the proximal end of support member 904a is secured to tubular member 910. Alternatively, the distal end of support member 904a can be secured directly to the distal end portion of guidewire tube 912. Support or feeler arm 906a is secured to support member 904a at the juncture of sections 904a1 and 904a2, which in the illustrative embodiment is at about the midpoint of sections 904a1 and 904a2, by any suitable means such as gluing or welding. Support member or feeler arm 906a is more flexible or less stiff than sections 904a1 and 904a2 and has at its free feeler end sensor 902a secured thereto. In the illustrative example, support member or feeler arm 906a can be in the form of a coil or spring as shown in FIGS. 28 and 29.

Support structure member 904b can comprise wire and includes proximal section 904b1 and distal section 904b2. The distal end of support member 904b is secured to tubular end member 908 and the proximal end of support member 904a is secured to tubular member 910. Alternatively, the distal end of support member 904b can be secured directly to the distal end portion of guidewire tube 912. Support member or feeler arm 906b is secured to support member 904b at the juncture of sections 904b1 and 904b2, which in the illustrative embodiment is at about the midpoint of sections 904a1 and 904a2, by any suitable means such as gluing or welding. Support member or feeler arm 906b is more flexible or less stiff than sections 904b1 and 904b2 and has at its free feeler end sensor 902b secured thereto. Support member or feeler arm 906b can be in the form of a coil or spring as shown in the example illustrated in FIGS. 28 and 29.

Guidewire tube 912, which is configured so that it can be tracked over guidewire 916, is secured to the end face 909 of tubular end member 908. End face 909 has a central opening to permit passage of guidewire 916 therethrough. Alternatively, end member 908 can be in the form of a collar that surrounds and is fixedly secured to the distal end portion of guidewire tube 912. When tubular members 908 and 910 are in the position shown in FIG. 28 where they are spaced from one another a distance L1, sections 904a1, 904a2, 904b1, and 904b2 are generally parallel to the longitudinal axis of tubular member 910 or guidewire 916. In this configuration, flexible members 906a and 906b also are generally parallel to the longitudinal axis of tubular member 910 or guidewire 916 or can be in a generally non-radially extended state as shown in FIG. 28.

Referring to FIG. 29, the relative position of tubular members 908 and 910 is changed (e.g., tubular member (guidewire tube) 912 is moved proximally (or retracted) to move tubular member 908 proximally as shown in FIG. 29) to reduce the distance between tubular members 908 and 910 to a distance L2. This causes axial compression of sections 904a and 904b to a position where sections 904a and 904b bend or flex outwardly or are radially expanded so that the distance between the flexible member attachments to members 904a,b changes from D1 to D2. Flexible members 906a and 906b are attached to the support structure in a manner so as to extend radially and proximally or away from the distal end of the apparatus as shown in FIG. 29.

When the sensors depicted in FIGS. 20-29 are electromagnetic coils with leads, the leads can extend from the sensors along their feeler arms and/or support, into the distal end of either tube 706, 802, or 910, between tubes 706 and 718, 802 and 810, or 910 and 912, and to the proximal region of the tubes where they are coupled to measuring unit 608 (FIG. 18).

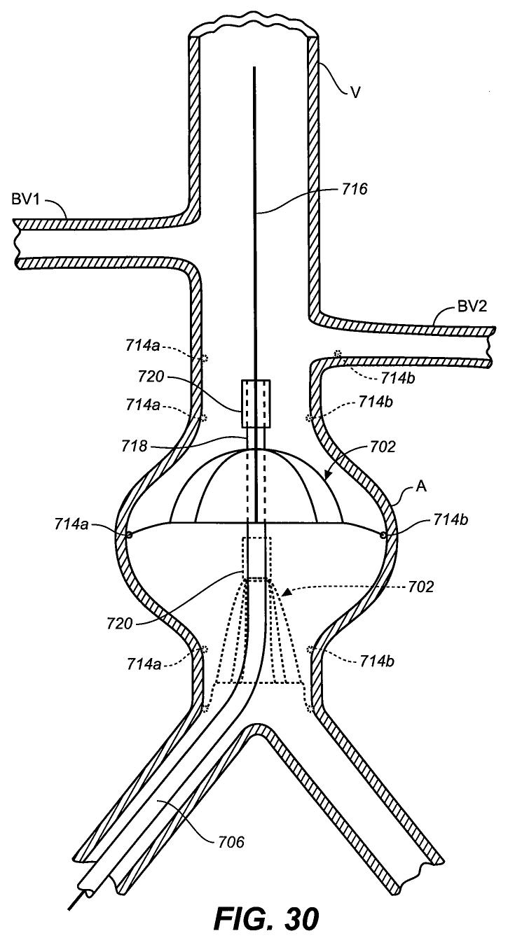

Referring to FIG. 30, one mapping procedure will be described, where for purposes of example, mapping and/or locator device 700 is used. In this procedure, device 700 is provided with four feeler arms each with an electromagnetic sensing coil (two feeler arm-sensing coil pairs are not shown for purposes of simplification) and device 700 is used to acquire data relating to anatomical features for generating an image of the contour of the target anatomy and/or provide diagnostic information without the assistance of a pre-operative scan (e.g., a pre-operative CT scan) or an intraoperative scan (e.g., a two-dimensional fluoroscopic scan).

The three magnetic field generators 302a,b,c are positioned on the operating table to facilitate triangulation of the exact position of each sensor in three-dimensional space using xyz coordinates as described above. The patient is prepared for surgery and a cut is made down to a femoral artery and guidewire 716 introduced. The operator tracks tubular member 706 of device 700 over guidewire 716 toward aneurysm A and branch vessels BV1 and BV2, which branch from vessel V, which in this example is the aorta. Restraint 720 is advanced to allow sensor support structure 702 and feeler arms 712a,b, and the two feeler arms not shown in FIG. 30 for purposes of simplification, radially expand as device 700 enters aneurysm A. In the radially expanded state, the sensors contact and track the inner wall surface of the vessel and aneurysm as shown in phantom and solid lines in FIG. 30.

The magnetic field generator is energized as described above and the sensors send signals to measuring unit 308 of circuit 300 indicative of their position in three-dimensional space as they are advanced through the vessel. Processor 306 can store the measured signals and/or process the measured signals to provide desired information. Processor 306 can determine the relative positions of the sensors in three-dimensional space and generate an image of that information in real time on display device 310 as they are advanced. In this manner, processor 306 can process the measured data and generate information that is sent to display device 310 to display an image of the contour of the inner wall of the vessel where the sensors have passed. Further, the acquired data can be processed or the image used to diagnose or size an unhealthy portion of the vasculature such as an abdominal aortic aneurysm. This information also can be used to select a prosthesis such as stent-graft, including its size, to be used to bypass (treat) the aneurysm.

Since in increase in surface area covered by the sensors improves image resolution and exactness of correspondence with the target vessel, the operator can rotate elongated member 706 about its longitudinal axis, while advancing the device to provide more coverage and data points. The additional data improves the exactness of the correspondence between the image and the actual vessel wall. Alternatively, or in combination with such rotation, the sensors also can be passed over the aneurysm more than once to provide more data points. In this case, each subsequent series of data points would be registered with the first series of data points. For example, a set of first pass data points corresponding to the bifurcation at the iliac arteries and a set of first pass data points corresponding to the lower wall portion of the ostium for branch vessel BV1 could be registered with corresponding points for each of the subsequent pass data points.

It also is noted that since device 700 has a much smaller profile and is more flexible than a conventional stent-graft catheter, it may not significantly distort the configuration or shape of the aneurysm and attendant vasculature as it is passed therethrough. The minimal amount or lack of vascular distortion due to device 700 also improves the accuracy of the imaging process.

After the sensors have reached the proximal landing of the aneurysm, the acquired data can be processed to generate an image of the contour of the aneurysm and to determine the size of the aneurysm to select a stent-graft of appropriate size and/or configuration. The length of the aneurysm can be determined based on the distance between the point where the sensors first move radially outward and the point after which they move radially inward and then exhibit little if any radial movement as they enter the proximal landing.

According to one variation, the sensors can be further advanced to acquire additional information. They can be further advanced and their position relayed to the operator via display 310 in real time and/or stored in processor 306 as the sensors move along the proximal landing to a point where one of the sensors moves radially outward in a manner indicative of entering branch vessel BV1 as described above and as shown in phantom in FIG. 30. The additional acquired data may be used in the stent-graft sizing step and/or determining if sufficient landing is present to secure the stent-graft below the lower branch vessel (BV1). In addition to providing the length of the proximal landing, processor 306 can process the data to provide the position of the opening to BV1 relative to the aneurysm and display an image of the aneurysm, proximal landing, and branch vessel ostium for BV1 on display device 310 in three-dimensions.

Alternatively, the operator can simply qualitatively track sensor radial movement as the sensor positions are displayed on monitor 310 in three-dimensional space as an indicator of the size of the aneurysm, proximal landing, and renal artery opening location.

In sum, the sensors can be moved to track any vasculature and provide position signals to measuring device 308 so that a three-dimensional model of the tracked vasculature can be displayed in three-dimensions. In this manner, the sensors map the contour of the vasculature.

After the desired data is gathered, restraint 720 is retracted, while holding elongated tubular member 706 stationary to radially compress support structure 702. With support structure 702 radially compressed, device 700 is withdrawn. Devices 800 and 900 are used in a similar manner.

In another approach, an intraoperative two-dimensional fluoroscopic scan is taken to provide a confirmation of branch vessel location. A contrast agent catheter is delivered through the femoral artery and the vasculature perfused with contrast and a fluoroscopic image including the renal arteries is taken and the acquired data input into processor or controller 306 where it can be stored and processed for display on display device 310 as a two-dimensional image. The fluoroscopic two-dimensional image will be used to provide a reference and confirm the results of the three-dimensional image generated by the sensors.

Device 700 is introduced into the femoral artery and advanced as described above. Processor or controller 306 processes the signals from the sensors as they are moved along the vessel wall inner surface to determine their position in three-dimensional space. The fluoroscopic scan data, which has been stored in processor 306, is registered with the sensor data using anatomical markers (e.g., the bifurcation at the iliac arteries and the lower renal vessel ostium). The angle of the fluoroscopic camera relative to the vasculature prior to the fluoroscopic scan also would be entered or stored in processor 306 to properly orient the two-dimensional fluoroscopic scan data points with the image generated from the acquired sensor location data, which identifies the position of the sensors in three-dimensional space. An image generated from the two-dimensional data points is overlayed on the three-dimensional data points and displayed on display device 310. The two-dimensional image would be displayed as an image slice showing different texture, color or border.

Any feature described in any one embodiment described herein can be combined with any other feature of any of the other embodiments whether preferred or not.

Variations and modifications of the devices and methods disclosed herein will be readily apparent to persons skilled in the art.

What is claimed is:

1. A method of locating a branch vessel in a human patient comprising;
tracking a sensor moving in a vessel along a first path;
detecting movement of the sensor away from the first path; and
determining if the detected movement is indicative of branch vessel entry.

2. A probe for locating or mapping structure in a patient comprising:
an elongated member configured for endovascular delivery in a patient, said elongated member having a proximal end portion and a distal end portion;
a first sensor coupled to said elongated member distal end portion;
a flexible member having a first portion and a second portion, said flexible member first portion being coupled to said elongated member distal end portion; and
a second sensor attached to said flexible member and suspended thereby.

3. The probe of claim 2 including a plurality of flexible members and a plurality of sensors secured to said flexible members, each flexible member extending from said elongated member distal end portion and having a feeler portion from which at least one of said sensors is suspended.

4. The probe of claim 3 wherein each flexible member is a wire.

5. The probe of claim 3 wherein each flexible member comprises shape memory material having a first memory set configuration from which it is deformable to a second configuration from which it tends to return toward said first configuration.

6. The probe of claim 3 further including a conductor extending from each of said sensors.

7. The probe of claim 3 wherein each of said sensors is a magnetic field sensing coil.

8. The probe of claim 2 further including a conductor extending from each of said sensors.

9. The probe of claim 2 wherein each of said sensors is a magnetic field sensing coil.

10. The probe of claim 2 wherein said elongated member is a catheter.

11. The probe of claim 2 wherein said flexible member has a fixed end portion and a feeler end portion, said flexible member fixed end portion being secured to said elongated member distal end portion.

12. The probe of claim 2 further including a support structure coupled to said elongated member, said first sensor being coupled to said support structure and to said elongated member through said support structure.

13. The probe of claim 12 wherein said support structure comprises a hoop and a plurality of support arms, each support arm having a first end attached to said hoop and a second end secured to said elongated member.

14. The probe of claim 13 wherein said flexible member has a fixed end portion and a feeler end portion, said flexible member fixed end portion being secured to said hoop, said second sensor being attached to said feeler end portion.

15. The probe of claim 14 including a second flexible member having a fixed end portion and a feeler end portion, said second flexible member fixed end portion being secured to said hoop, said second sensor being attached to said feeler end portion.

16. The probe of claim 12 including a tubular restraint slidably mounted over the elongated member, said support structure having a memory shape and said tubular restraint being arranged such that when moved in one direction it moves over the support structure to radially compress the support structure and when moved in another direction it releases said support structure allows the support structure to move radially outward toward said memory shape.

17. The probe of claim 16 wherein said elongated member is tubular, and further including a guidewire tube slidably disposed in said elongated member, said guidewire tube having a distal end portion secured to said tubular restraint.

18. The probe of claim 2 further including a tubular restraint slidably mounted over the elongated member, said flexible member having a memory shape and said tubular restraint being arranged such that when moved in one direction it moves over the support structure to radially move the flexible member toward said elongated member and when moved in another direction it releases said flexible member and allows the flexible member to move radially outward.

19. The probe of claim 18 wherein said elongated member is tubular, and further including a guidewire tube slidably disposed in said elongated member, said guidewire tube having a distal end portion secured to said tubular restraint.

20. The probe of claim 2 further including a support structure and a guidewire tube, said support structure having a first portion attached to said elongated member and a second portion coupled to said guidewire tube, said first sensor being coupled to said support structure and to said elongated member through said support structure.

21. The probe of claim 20 wherein said flexible member is attached to said support structure, further including a second flexible member attached to said support structure, said first sensor being attached to said second flexible member.

22. The probe of claim 20, wherein said support structure comprises a wire having an intermediate portion that moves radially outward when the wire is axially compressed.

23. The probe of claim 22 further including a collar, said collar being secured to a distal portion of said guidewire tube and said support structure being secured to said guidewire through said collar.

24. The probe of claim 2 wherein said flexible member has a constant flexibility along its longitudinal axis.

25. The probe of claim 2 wherein said flexible member as a varying flexibility along its longitudinal axis.

26. The probe of claim 2 wherein said flexible member has sections having different flexibility.

27. The probe of claim 2 wherein said flexible member comprises a wire coil.

28. A method of mapping the contour of an inner surface of a vessel wall in a patient comprising:
advancing a plurality of sensors along an inner surface of a vessel wall in a patient;
acquiring data indicative of the position of the sensors in three-dimensional space as they are advanced along the surface; and
processing the acquired data to generate a three-dimensional image corresponding to the contour of a portion of the inner vessel surface.

29. The method of claim 28 wherein the sensors are passed over an aneurysm and the acquired data is processed to generate a three-dimensional image corresponding at least in part to the aneurysm.

30. The method of claim 29 wherein a stent-graft for treating the aneurysm is selected based on the acquired data.

31. The method of claim 28 wherein the sensors are passed over an aneurysm and a stent-graft for treating the aneurysm is selected based on the acquired data.

32. The method of claim 28 wherein at least four sensors suspended from a support at different positions are advanced along the inner surface.

33. The method of claim 28 wherein the sensors are suspended about a support and manipulated to make multiple passes along the same portion of the vessel, and the support is rotated before each pass made after the first pass.

34. The method of claim 28 wherein the sensors are moved around the longitudinal axis of the guidewire as they are advanced and data corresponding to their position as they are advanced acquired.

35. The method of claim 28 wherein the sensors are suspended about a support and the support is rotated as the sensors are advanced along the inner surface.

36. The method of claim 28 wherein the sensors are electromagnetic sensing coils.

37. A method of mapping the contour of an inner surface of a vessel wall in a patient comprising:
advancing a sensor along the inner surface of a vessel wall in a patient in both a circumferential and axial direction;
acquiring data indicative of the position of the sensor in three-dimensional space as it is advanced along the surface; and
processing the acquired data to generate a three-dimensional image corresponding to the contour of a portion of the inner vessel surface.

38. The method of claim 37 wherein the sensor is passed over an aneurysm and the acquired data is processed to generate a three-dimensional image corresponding at least in part to the aneurysm.

39. The method of claim 38 wherein a stent-graft for treating the aneurysm is selected based on the acquired data.

40. The method of claim 37 wherein the sensor is passed over an aneurysm in the vessel and a stent-graft for treating the aneurysm is selected based on the acquired data.

41. The method of claim 37 wherein the sensor is moved along a spiral path.

42. The method of claim 37 wherein a plurality of sensors are advanced along the inner surface of the vessel wall and data acquired indicative of the position of the sensors in three-dimensional space as they are advanced along the surface.

43. The method of claim 37 wherein at least four sensors are advanced along the inner surface of the vessel wall.

44. The method of claim 37 wherein the sensors are electromagnetic sensing coils.

45. A method of selecting vascular prosthesis comprising:
advancing a sensor along an inner surface of a vessel wall;
acquiring data indicative of the position of the sensor in three-dimensional space as it is advanced along the inner surface; and
selecting a prosthesis based on the acquired data.

46. The method of claim 45 wherein the sensor is passed over an aneurysm in the vessel wall and a stent-graft for treating the aneurysm is selected based on the acquired data.

47. The method of claim 46 wherein the sensors are electromagnetic sensing coils.
